(19) United States
(12) Patent Application Publication
(10) Pub. No.: US 2006/0013895 A1
(43) Pub. Date: Jan. 19, 2006

(54) Use of a composition and a cleaning tablet containing said composition for disinfecting purposes

(76) Inventor: Peter Wollwage, Mauren (LI)

Correspondence Address: NIXON & VANDERHYE, PC, 901 NORTH GLEBE ROAD, 11TH FLOOR, ARLINGTON, VA, 22203, US

(21) Appl. No.: 10/518,908

(22) PCT Filed: Jun. 24, 2003
(86) PCT No.: PCT/CH03/00421

§ 371 Date: Sep. 1, 2005

(30) Foreign Application Priority Data
Jun. 24, 2002 (CH) 1079/02
Mar. 24, 2003 (CH) 0509/03

(51) Int. Cl. A61K 33/14

(52) U.S. Cl. 424/661; 424/723

(57) ABSTRACT

The invention concerns the use of a composition containing at least one organic or inorganic compound or substance combination that releases chlorine or bromine in aqueous solution, as well as
whenever appropriate, a surface-active substance (tenside) or substance mixture, aromatic materials, adjuvants and binders for the disinfection of dental objects such as toothbrushes, dentures or razors and the like contaminated by retro or herpes viruses, pseudomonas aeruginosa and/or candida or parts of the body that have been attacked by retro or herpes viruses, pseudomonas aeruginosa and/or candida.

### TECHNICAL AREA OF INVENTION

The present invention concerns the use of a composition and a cleaning tablet containing the composition for disinfection, especially of dental objects and for the local treatment of body parts affected by micro-organisms, namely by candida or retro or herpes viruses.

### PRIOR ART

It is known that tooth brushes can shelter bacteria, viruses and other organisms that cause illnesses. Tooth brushes may also be contaminated with germs, since these are inevitably present in bathrooms. Every time the toilet is flushed, dangerous germs are propelled into the air. Such germs can also contaminate toothbrushes, which are often kept in glasses with the brush pointing upwards.

Toothbrushes are already dirty, even if they are used only for a few days. It was possible to demonstrate that bacteria, viruses and other germs can easily survive 24 hours. Since several toothbrushes are often kept practically next to each other, there is also a risk of cross-contamination.

Illness-causing bacteria and viruses can therefore contaminate the tooth brushes of other family members. Consequently, toothbrushes should be changed rather often and, in any case, after an illness.

For the aforementioned reasons, it has already been suggested that toothbrushes should be cleaned from time to time in dish washing machines. This is a simple way of sterilizing toothbrushes at home. When travelling, however, a dish washer is not normally available.

EP-A-0 081 962 discloses a self-dissolving tablet for the cleaning of artificial dentures. The tablet contains a perborate compound that releases oxygen and a chloroisocyanurate compound that releases hypochlorite when the tablet comes into contact with water. The tablet also contains alkaline compounds that assure a pH-value of approximately 11 when the tablet is dissolved in the aqueous medium.

U.S. Pat. No. 3,936,385 also discloses a self-dissolving cleaning tablet containing a peroxo compound that releases oxygen when in contact with water and a chlorine compound that, likewise when in contact with water, releases hypochlorite, where the hypochlorite/oxygen ratio amounts to at least 1.1:1. Furthermore, the table contains an alkaline compound in such a quantity as to produce a pH-value greater than 10.5.

A disadvantage of the aforementioned tablets is due to their chlorine smell, which is produced when these hypochlorite releasing tablets are dissolved. EP-A-0 164 472 therefore suggests that a hypochlorite deactivator should be added to the composition. For example, sodium perborate monohydrate and sodium nitrite are used as deactivators, both of which are capable of reacting with the hypochlorite. The deactivator is released with a certain delay, so that the hypochlorite can at first produce its effect. As a solution for the delayed release of the deactivator it is suggested that the tablet should consist of two areas with different compositions and solution properties. The first area of the tablet contains the cleaning compound, while the second, the area that dissolves more slowly, contains the deactivator. The pH-value of the cleaning solution of these tablets once again amounts to at least 7.5 and preferably to between 8.5 and 11. A similar solution is also described by EP-A-0 360 299. The aforesaid tablets are difficult to produce and therefore costly.

With a view to improving the cleaning effect, EP-A-0 451 105 suggests the use of a cleaning tablet consisting of two parts with different compositions in which the tablet parts or areas are not arranged one above the other, but next to each other. This has the advantage that the tablet, quite independently of its position, will always dissolve at the same speed. In case a pH-change is desired during the dissolution, EP-A-0 451 105 suggests that an acid compound should be mixed into one part of the tablet and a basic (neutralizing) compound into the other part of the tablet. The acid component can initially assure a pH-value of the cleaning solution of less than 2.5. However, due to complete and retarded dissolution of the basic compound, the pH-value will then be raised to at least 5.5 or more, i.e. in the extreme case the total of the acid components contained in the tablet are only slightly in excess as compared with the basic components, in the normal case however they are substantially greater, as is the case of the known cleaning tablets. The principal components of the tablet of EP-A-0 451105 can be acid and basic compounds, oxidizing agents, bleaching means, chelate formers, surface-active substances, lubricants and additives. Among others, hypochlorite-forming substances or substance combinations that release or form hypochlorite when in contact with water are suggested as bleaching materials. Among others, a mixture of sodium chloride and sodium persulphate is named as an example of such a material combination. Nevertheless, it is not a declared aim of EP-A-0 451 105 to generate chlorine in situ from sodium chloride and sodium persulphate. Furthermore, it is known that an alkaline environment should be available for the formation of hypochlorite. Nor does EP-A-0 451 104 make any specific statements about the specific effectiveness of the compositions suggested by way of example.

According to EP-A-0 451 105, a multi-part tablet with areas of different compositions may contain a multitude of different substances, but only one concrete embodiment is reproduced in the description. This composition example does not contain any saline chlorine or bromine compounds with a strong oxidizing agent and an acid or acid excess with the simultaneous presence of basic substances.

It is known from WO 97/19708 the compounds releasing chlorine have a bactericidal effect. Such compounds have for a long time been used, for example, for cleaning and disinfecting dishes and crockery for children. WO 97/19708 suggests an improved composition that produces a good cleaning and disinfection effect. The composition contains an anionic detergent, a phosphate builder, borax, an alkali metal chloride or sulphate, as well as a compound releasing chlorine. According to WO 97/19708, the addition of an alkali metal chloride contributes to increasing the long-term stability of the chlorine releasing compound. The composition of WO 97 /19708 proved to be effective for killing choli bacteria and staphylococci.

It is known that the fungus candida albicans can cause painful areas on the palate and in the area of the throat. Hitherto, however mouth hygiene has paid little attention to this aspect. Investigations have also shown that that a number of other illness-causing micro-organisms can at times occur on removable dentures. For example, gram-negative bacteria that can lead to endotoxic sepsis and systemic illness have been found.

Although the known, self-dissolving cleaning tablets obtainable in commerce are claimed to have a disinfecting cleaning effect, their fungicidal effect leaves much to be desired, especially in the case of candida. Tests have shown that traditional tablets are not capable of effectively reducing candida. Furthermore, it is not known that retro or herpes viruses are rendered harmless by known cleaning tablets.

### OBJECT OF THE INVENTION

It is the object of the present invention to suggest a means for cleaning and disinfecting artificial dentures, toothbrushes and objects and body parts affected by micro-organisms like candida or retro or herpes viruses. A further object of the invention is to make available a means that can be handled in a simple manner, can readily be used when travelling and occupies little space. Yet another object is to make available a means with which bacteria like streptococci and pseudomonas, fungi like candida albicans, as well as viruses, especially herpes viruses, can be effectively rendered harmless. It is another object to suggest a pharmaceutically effective composition for rendering candida harmless, especially the fungus candida albicans.

### DESCRIPTION OF THE INVENTION

Object of the present invention is the novel use of a composition containing
at least one organic or inorganic compound that releases chlorine or bromine or a substance combination that generates chlorine or bromine when in aqueous solution, as well as
possibly a surface-active substance (tenside) or substance mixture, aromatic substances, adjuvants and binding agents,
   for the disinfection of such dental objects as toothbrushes, dentures or razor sets and the like contaminated with retro or herpes viruses and/or candida or of body parts affected by retro or herpes viruses and/or candida. To the surprise of the inventor, chlorine and bromine are capable of rendering harmless even retro or herpes viruses and/or candida. This result is surprising inasmuch as known compositions available in the form of self-dissolving cleaning tablets are not capable of killing candida.

According to a particularly advantageous embodiment variant, the substance combination comprises saline chlorine or bromine compound and at least one suitable oxidizing agent for the in situ production of chlorine and/or bromine during the dissolution of the composition in an aqueous solution. The substance combination is particularly effective, because chlorine or bromine are formed in situ.

Advantageously, the composition will contain an acid. To the surprise of the inventor, it has been found that in an acid environment, i.e. at a pH-value less than 7.0 the effectiveness of the composition becomes clearly better.

Advantageously, the acid will be contained in the composition in such a quantity that the composition, when completely dissolved in a certain quantity of aqueous solution, will have a pH-value less than 6, preferably less than 5.5 and even more preferably less than 5.0. Such a composition has the advantage that even staphylococci, streptococci and pseudomonas will be rendered harmless in a very brief time. To the surprise of the inventor, it has been found that in an acid environment a clearly better fungicidal effect can be obtained—both as regards the effectiveness range and as regards the effectiveness—than at a pH-value greater than 7, as is the case with conventional compositions that have been known for a long time. At a pH-value of less than 5.5, in particular, it is possible to kill the fungus candida albicans practically completely within the space of a few minutes. The composition may be made available as a solid mixture or as an aqueous composition.

In the initial compound the chlorine or bromine atom may be available in the form of an alkali or alkaline earth metal salt or in some other compound that will release chlorine and/or bromine ions in aqueous solution or linked with an organic rest. Chemical compounds that can release chlorine as, for example, chloramin B (N-chlorbenzenesulphonamido-sodium), chloramin T(p-toluenesulphonchloramin-sodium) dichloramin T (p-toluenesulphondichloramid), halazon (p-dichlorsulphamyl benzoic acid), dichlorcyanic acid, trichlorcyanic acid, TCM (trichlormelamin), 1,3-dichlor-5,5-dimethylhydantoin, dichlorglycoluril, succinchlorimid or chloroazodin (N,N′-dichlorodiazodicarbonamidin). These compounds can be used either by themselves or in any desired mixture. The previously mentioned compounds can develop an antiviral and fungicidal effect in an acid environment.

Advantageously, the substance combination generating the chlorine or the bromine will be a chlorine or a bromine compound in the form of an alkali, alkaline earth or other metallic salt and a suitable oxidizing agent. In combination with at least one suitable oxidizing agent, chlorine (Cl2) or dichloroxide (Cl2O—) and bromine and/or bromine oxide, respectively, can be formed by reaction with the chlorine (Cl—) or bromine ions present in the solution. Different compositions and substance combinations known to the person skilled in the art can be used for the in situ chlorine and/or bromine production. The advantage of such a composition is that the chlorine or the bromine is released relatively quickly and therefor produces a quick effect. If the desired reaction is to be obtained, it is self-evident that at least in the preferred pH-range of less than 6, respectively less than 5.5, the oxidation potential of the oxidizing agent in solution should be higher than the oxidation potential of Cl1-/Cl0 and/or Br1/Br0.

Advantageously, the composition will contain further fungicidal compounds, such as, for example, potassium monosupersulphate, potassium carboate, sodium peroxycarbonate, sodium benzoate, subtilisin, potassium benzoate, chlorhexidine, a combination of chlorhexidine and thymol, cetylpyridine chloride, such halogen releasing compounds as PVP-iodine and cyanuric chloride and/or such compounds releasing formaldehyde as paraformaldehyde and/or methylol compounds, etc.

According to a preferred embodiment, the composition is a mixture of solids. With a view to improving the dissolution and the uniformity of the solution, the composition will advantageously contain effervescing salts, i.e. means for accelerating the dissolution, which in English are also often known as “effervescents”. The means for accelerating the dissolution are, for example, a compound containing a carbonate (CO32-) or bicarbonate (HCO₃₋), say sodium carbonate or sodium bicarbonate, and an acid. For example, carbon acids, especially bicarbonic acids, can be used as acids, or also every other acid known to the person skilled in the art that is preferably physiologically safe. Use is preferably made of acids that will readily decompose in nature.

Although basically different oxidation agents can be used, the composition will advantageously contain a hydrogenperoxosulphate compound or a persulphate compound or hydrogen peroxide as oxidation agent. An advantageous composition that can be produced at a favourable cost contains potassiumhydrogenmonopersulphate (KHSO₅) , cooking salt (NaCl) and an acid, preferably in the form of a mono, di- or tricarbon acid as oxalic acid, tartaric acid, succinic acid or citric acid. Preferably, the quantity of acid contained in the composition is such that, following the dissolution of the composition in the aqueous environment a pH-value of less than 5.5, preferably less than 5 will be obtained. Citric acid is physiological perfectly safe, is quickly decomposed in nature and is therefore preferably used in the composition.

A particularly good disinfection and cleaning effect is obtained when the composition also contains at least one suitable tenside. Care must however be taken to assure that the utilized tenside is compatible with the disinfection means. Fundamentally it is possible to use different tensides known to the person skilled in the art. But it is of importance that the tensides are stable at a pH-value smaller than 8, preferably smaller than 7. In this connection “stable” is to be understood in the sense that a maximum of 10% of the employed tenside will decompose in solution at room temperature within 30 minutes. In combination with the composition in accordance with the invention, it is preferred if the employed tenside is not precipitated or decomposed at a pH of 6 to 4, preferably a pH smaller than 5.5. Preferably, fatty alcohol polyglycol ethers, alkyl benzenesulphonates and alkyl sulphonates. Anionic tensides, preferably of the group of alkyl ether sulphates, such as fatty alcohol ether sulphate alkali salts, for example sodium-n-alkyl-C₁₂₋₁₄-diglycol ether sulphate, fatty acid amido propyl Betain and/or sodium lauryl sulphosuccinate or sodium-n-alkyl-C₁₂₋₁₄-diglycol ether sulphate have proved to be particularly successful. Also suitable are, for example Na-lauryl sulphate, Na-lauryl sulpho acetate and trisodium phosphate. Preferably, tensides (powders) available as solids at room temperature are used in the composition.

The composition is advantageously made available in the form of tablets or granulates. Tablets and granulates can be readily portioned and taken on voyages. The composition can be dosed in such a manner that an effective fungicidal solution is brought into being upon the dissolution of the tablet or a package of granulate in a glass of water. Advantageously, the composition will contain a binder and, optionally, aromatic and colouring substances and adjuvants such as materials for dehardening the water, fillers and the like. For example, modified maize starch, microcrystalline cellulose, sorbito, hydrated soya triglycerides, polyethylene glycol such as PEG 180, PEG 150, PEG 75, polyvinyl pyrolidone or a copolymer of polyvinyl pyrolidone vinyl acetate or such sugar derivatives as lactose or combinations of the aforementioned compounds may be used as binders. The share by weight of the binder amounts advantageously to a maximum of about 30% and lies preferably between 5 and 25%.

Advantageously, the share by weight of the components that accelerate the dissolution will lie in the range between 15 and 40%, preferable 15 and 30%, of the total weight of the tablet. Advantageously, the employed quantity of the tenside (percent by weight) will exceed the employed quantity of the bactericide (percent by weight).

The composition in accordance with the invention is used for disinfecting dental objects, especially toothbrushes and dentures, razors and the like, as also objects for body care such as foot and hand baths. It is particularly important that candida, especially, candida albicans, which is often the cause of infections of the mouth cavity, can be killed. Candida is often found also on dentures and occasionally on toothbrushes, so that fighting against them is of special importance for human hygiene. Of particular importance is also pharmaceutical use of the composition, for example, for the production of a means for treating parts of the body affected by candida.

Object of the invention is also the use of the composition for the production of a means for the disinfection of surfaces contaminated by candida, retro and herpes viruses, especially the mucous membranes of the mouth cavity. The composition has proved effective in fighting herpes viruses, especially human herpes virus 1 (HHV-1; herpes simplex virus), family Herpesviridae, subfamily alphaherpesviridae, Human herpes virus 5(HHV-5) Human cytomegalovirus strain Toledo, family Herpesviridae, subfamily betaherpesvirinae, but also retro viruses such as those of the family Retroviridae (Spumavirus genus).

But the composition is particularly suitable also for fighting parts of the body or objects contaminated with staphylococci, especially staphylococci aureaus, streptococcus pyogenes, streptococcus mutans, escherida coli, pseudomonas aeruginosa, simian foamy virus (SFV-bab) strain OCOM1-26.

Another object of the invention is the use of the composition for the production of a means for the local treatment of painful parts and infections of the palate and the pharynx area caused by candida, especially candida albicans.

Yet another object is the use of the composition for the production of a preparation for the local treatment of parts of the body or object affected by retro or herpes viruses.

A further object is the use of the composition for the production of a preparation for the local treatment of fungus affection on the human or animal body, especially foot and nail fungi.

### EXAMPLE

An example of a composition contains the following components:

| | in percent by |
|---|---|
| Substance | weight (%) |
| Potassium hydrogen | 5 |
| monopersuiphate | |
| Na-lauryl sulphate | 15 |
| Sodium bicarbonate | 20 |
| Citric acid | 30 |
| Binder | 20 |
| Sodium chloride | 10 |

The effect of compositions in accordance with the invention was tested by means of the following mixture:

| | |
|---|---|
| Streptococcus pyogenes ATCC | |
| 19615 | |
| Streptococcus mutans ATCC | |
| 25175 | |
| Pseudomonas aeruginosa | |
| Candida albicans | |
| Escherichia coli ATCC 25922 | RT |
| Initial concentration: | 10⁵-10⁶ CFU/ml |

The micro-organisms named above were established in each case on two toothbrushes, which were then dipped into a test solution. The test solution was produced by dissolving half a tablet in 100 ml of water at a temperature of 21° C. An entire tablet weighed 2.5 g, and half a tablet therefore 1.25 g. It was found that the bacteria/fungus mixture was already killed after 5 minutes. The final concentration of the micro organisms amounted in each case to less than 10¹.

In another series of tests several small pieces of a denture (methyl methacrylate) were placed in a nutrient solution that had been inoculated with test micro-organisms. The nutrient solution had an initial concentration of about 10⁶ micro-organisms per millilitre. Subsequently the nutrient solution was shaken for a total period of about 12 hours at a heightened temperature and also ventilated. Following the first 6 hours, the solution was decanted and fresh nutrient solution was added. The liquid was then poured away and the denture pieces dried in Petri dishes.

The denture pieces were then divided into two groups, where the first group was placed for 5 minutes into sterile water, and the second group into a composition in accordance with the invention. The effectiveness was then tested by determining the number of micro-organisms on the denture surface.

A high fungicidal effect was noted especially with the following micro-organisms:

Candida, especially candida albicans, herpes viruses, staphylococci, especially staphylococcus aureus, gram-negative bacteria such as pseudomonas aeruginosa, herpes viruses, especially human herpes virus 1(HHV-1; herpes simplex virus), family herpesviridae, subfamily alphaherpesviridae, human herpes virus 5(HHV-5) human cytomegalovirus strain Toledo, family herpesviridae, subfamily betaherpesvirinae, simian foamy virus (SFV-bab), such retro viruses as family retroviridae (spumavirus genus).

For disinfection purposes, a denture affected by micro-organisms is subjected for a certain period of time, typically five minutes, to the action of a substance mixture that releases chlorine or chlorine oxide and/or bromine or bromine oxide. To this end an effervescent cleaning tablet is placed together with the denture that is to be disinfected into an empty glass, which is then filled with water. The aqueous solution is mixed while the tablet dissolves. At the same time chlorine and bromine, respectively, are released or formed in situ. Due to the agitation, chlorine and bromine, respectively, arrive at all parts, so that a good disinfecting effect is obtained.

The invention concerns the use of a composition containing
at least one organic or inorganic compound or substance combination that releases chlorine or bromine in aqueous solution, as well as
whenever appropriate, a surface-active substance (tenside) or substance mixture, flavouring agents, adjuvants and binders for the disinfection of dental objects such as toothbrushes, dentures or razors and the like contaminated by retro or herpes viruses, pseudomonas aeruginosa and/or candida or parts of the body that have been attacked by retro or herpes viruses, pseudomonas aeruginosa and/or candida.

1-17. (canceled)

18. Use of composition containing
at least one saline chloride compound in the form of a salt of an alkali or an alkaline earth metal and at least one suitable oxidation means for the in situ production of chlorine during the dissolution of the composition in an aqueous solution, where the oxidation potential of the oxidation means in an aqueous solution is higher than the oxidation potential of Cl¹/C1° at least in the claimed range,
an acid in such quantity that the composition dissolved in a certain quantity of an aqueous solution will produce a pH-valve of less than 6, preferably less than 5.5 and even more preferably less than 5, as well as
whenever appropriate, a surface-active substance (tenside) or substance mixture, aromatic substance adjuvants and binders,
for the disinfection of dental objects such as toothbrushes, dentures and the like contaminated by candida or parts of the body that have been attacked by candida.

19. Use in accordance with claim 18, characterized in that the composition is available in the form of a mixture of solids, especially a granulate or a tablet.

20. Use in accordance with claim 18, characterized in that the composition contains means for accelerating the dissolution (effervescent salts—effervescents), for example a compound containing a carbonate or bicarbonate such as, for example sodium carbonate or sodium bicarbonate, and at least a stoichiometric quantity of acid for the production of a pH-value of less than 7 is contained in the composition.

21. Use in accordance with claim 18, characterized in that a hydrogen peroxosulphate compound is contained as oxidation means.

22. Use in accordance with claim 18, characterized in that the composition contains at least potassium hydrogen peroxosulphate as oxidizing agent, a saline chlorine compound, e.g. cooking salt (NaCl) as chloride compound and a carbon acid, preferably a mono-, di- or tricarbon acid, such as tartaric or citric acid, for the production of an acid pH-Value in the dissolved composition.

23. Use in accordance with claim 18, characterized in that the composition contains a binder and, optionally, aromatic and colouring substances and adjuvants such as substances for softening the water, fillers and the like, and is available in tablets or granulate form.

24. Use in accordance with claim 18, characterized in that the composition contains at least one tensed (surface-active substance or substance mixture).

25. Use in accordance with claim 18, characterized in that the composition is available in the form of a self-dissolving cleaning tablet.

26. Use in accordance with claim 25, characterized in that the composition or cleaning tablet has a homogeneous composition.

27. Use in accordance with claim 25, characterized in that the composition or the cleaning tablet is dissolved in an aqueous solution, preferable in water, in the presence of the object or body part that is to be disinfected.

28. Use in accordance with claim 18, characterized in that the binder is a copolymer of ethylene oxide and propylene oxide, polyvinyl pyrolidon or a copolymer of polyvinyl pyrolidon and vinyl acetate.

29. Use of a composition containing
at least one saline chloride compound in the form of salt of an alkaline or alkali earth metal and at least one suitable oxidizing agent for the in situ production of chlorine during the dissolution of the composition in an aqueous solution, where the oxidation potential of the oxidizing agent in aqueous solution is greater than the oxidizing potential of C1¹⁻/C1⁰ at least within the claimed pH-range,
an acid in such quantity that the composition dissolved. in a certain quantity of an aqueous solution produces a pH-value of less than 6, preferably less than 5.5 and even more preferably less than 5.0, as well as
whenever appropriate, a surface-active substance (tenside) or substance mixture, aromatic substances, adjuvants and binders,
for the production of a pharmaceutically effective means for treating body parts contaminated or affected by candida albicans.
